(19) United States
(12) Patent Application Publication
(10) Pub. No.: US 2026/0284089 A1
(43) Pub. Date: Sep. 24, 2026

(54) COMPOSITIONS AND METHODS FOR IMPROVING METABOLIC HEALTH

(71) Applicant: SOCIÉTÉ DES PRODUITS NESTLÉ S.A., Vevey (CH)

(72) Inventors: Stefan CHRISTEN, Ecublens (CH); Jean-Philippe GODIN, Ecublens (CH); Olga SAKWINSKA, Pully (CH); Maria Pilar GINER, Lausanne (CH); Léa SIEGWALD, Epalinges (CH); Anna CHERTA, Préverenges (CH); Claire Laurence Lucie Marie BOULANGE, Oron-la-Ville (CH)

(21) Appl. No.: 19/479,014

(22) PCT Filed: Apr. 29, 2024
(86) PCT No.: PCT/EP2024/061792

§ 371 Date: Oct. 27, 2025

Related U.S. Application Data
(60) Provisional application No. 63/462,788, filed on Apr. 28, 2023.
(60) Provisional application No. 63/522,804, filed on Jun. 23, 2023.

(51) Int. Cl. A61K 31/702; A23C 9/20; A61K 47/26; A61K 47/42

(52) U.S. Cl. CPC A61K 31/702; A23C 9/203; A61K 47/26; A61K 47/42

(57) ABSTRACT

Methods and compositions improve metabolic health. A method for improving metabolic health includes administering to an individual in need thereof a composition comprising milk protein, preferably 20-40% by dry weight of the composition; and a mixture of lactose, glucose, galactose and galactooligosaccharide, preferably 35-55% by dry weight of the composition. The mixture of lactose, glucose, galactose, and galactooligosacchride can contain 0.1-30% by weight of the lactose, 8-40% by weight of the glucose, 0.1-20% by weight of the galactose and 40-90% by weight of the galactooligosaccharide.

### TECHNICAL FIELD

The present disclosure generally relates to compositions and methods for improving metabolic health. Some embodiments are directed to a composition comprising milk protein and further comprising a mixture of lactose, glucose, galactose and galactooligosaccharide (GOS).

### BACKGROUND

Gut microbiota metabolites play an important role in host health and nutrient metabolism. More and more scientific evidence suggest that metabolites produced by gut microbiota are important mediators of benefits for the host. Supplementation with galactooligosaccharides (GOS) has been previously linked to several health benefits, predominantly via gut microbiome modulation. While many studies are focused on beneficial metabolites originating from gut carbohydrate fermentation, it appears that bacterial metabolites from protein metabolisms also play an important role in host physiology.

There remains a need for a composition for easy and convenient administration to an individual resulting in an increase in beneficial microbial metabolites.

### SUMMARY

Embodiments of the disclosure concern methods and/or compositions for improving metabolic health, in which reduced blood or intracellular levels of certain metabolites, and the fecal level of certain bacteria are directly or indirectly related affected. In specific embodiments, the level of certain metabolites, and corresponding level of certain bacteria such as Bifidobacteria, in an individual is increased, upon which a medical condition or physical state is thereby treated, prevented, or had a delay in onset. In certain embodiments, the level of certain metabolites in an individual is reduced, upon which a medical condition or physical state is thereby treated, prevented, or has a delay in onset.

In particular embodiments, a method for improving metabolic health includes administering to an individual in need thereof a low sugar, low lactose, milk-based composition comprising galactooligosaccharide. In some embodiments, the composition comprises milk protein, preferably at 20-40% by dry weight of the composition; and a mixture of lactose, glucose, galactose and galactooligosaccharide, the mixture preferably at 35-55% or 32-55% by dry weight of the composition; preferably, the mixture comprising: 0.1-30% by weight of the lactose, 8-40% by weight of the glucose, 0.1-20% by weight of the galactose and 40-90% by weight of the galactooligosaccharide.
The improving of the metabolic health can comprise at least one result selected from the group consisting of: (a) appetite or food intake control or regulation; (b) managing glucose levels; (c) weight management; (d) normalizing blood lipid profiles; (e) skin health; (f) reducing inflammation; (g) preventing and/or attenuating oxidative stress; (h) improved lipid metabolism; (i) improving intestinal barrier; (j) reducing intestinal permeability; (k) modulation of glucose metabolism; (l) protection from fatty liver; (m) promoting growth of Faecalibacterium prausnitzii, (n) protecting kidneys, particularly in chronic kidney disease; (o) promoting growth of Bifidobacterium; (p) increasing Bifidobacterium shunt pathway; (q) increasing NAD salvage pathway; (r) preservation of β-cell function; (s) preventing diabetes, (t) improving insulin sensitivity; (u) preventing and/or treating obesity; (x) regulating blood pressure; (y) maintaining insulin response; and (z) combinations thereof.

The composition may further comprise 0.5-30% by dry weight of at least one of milk fat or mineral derived from milk, preferably a combination thereof. The composition may further comprise 5-20% by dry weight of vegetable oil.

Preferably the composition is administered in an amount effective to increase one or more metabolite levels selected from the group consisting of: 3-indole propionate, propionate, indoxyl-3-carboxylate, valerate, tricarballylic acid, imidazole acetate, acetate, N,N,-dimethylglycine, caprylic acid, nicotinamide, beta-alanine, ketone bodies, 3-hydroxybutyrate, 12,13-diHODE, and/or 3-hydroxydecanoic acid. Preferably, the composition is administered in an effective amount to increase one or more Bifidobacterium, including but not limited to B. longum, B. adolescentis, B. bifidum.

Another aspect of the present disclosure is a method for one or more of preventing oxidative stress, preventing and/or treating type II diabetes, reducing inflammation, improving the intestinal barrier, reducing intestinal permeability, modulating glucose metabolism, improving insulin sensitivity, preventing obesity, appetite regulation, and/or protecting against fatty liver, the method comprising administering to an individual in need thereof or at risk thereof any of the compositions disclosed herein. The composition may be administered in an amount effective for increasing levels of 3-indole propionate in the individual. The composition may be administered to the individual in a unit dosage form, wherein the unit dosage form comprises an amount of the composition effective for increasing levels of 3-indole propionate in an individual.

Yet another aspect of the present disclosure is a method for preventing and/or treating kidney disease, the method comprising administering to an individual in need thereof or at risk thereof any of the compositions disclosed herein. The composition may be administered in an amount effective for increasing levels of 3-indole propionate in an individual and decreased levels of at least one metabolite selected from the group consisting of p-cresol sulfate and indoxyl sulfate. The composition may be administered to the individual in a unit dosage form, wherein the unit dosage form comprises an amount of the composition effective for increasing levels of 3-indole propionate and decreasing levels of at least one of metabolite selected from the group consisting of p-cresol sulfate and indoxyl sulfate in the individual.

Yet another aspect of the present disclosure is a method for one or more of treating or preventing pellagra, acne, or skin cancer, improving skin health, and/or improving the level of blood lipids, the method comprising administering to an individual in need thereof or at risk thereof any of the compositions disclosed herein. The composition may be administered in an amount effective for increasing levels of nicotinamide in the individual. The composition may be administered to the individual in a unit dosage form, wherein the unit dosage form comprises an amount of the composition effective for increasing levels of nicotinamide in an individual.

Yet another aspect of the present disclosure is a method for one or more of preventing and/or treating obesity, improving appetite control, improving food intake control, and/or managing weight, the method comprising administering to an individual in need thereof or at risk thereof any of the compositions disclosed herein. The composition may be administered in an amount effective for increasing levels of acetate in the individual. The composition may be administered to the individual in a unit dosage form, wherein the unit dosage form comprises an amount of the composition effective for increasing levels of acetate in an individual.

Yet another aspect of the present disclosure is a method for one or more of preventing and/or treating obesity, improving appetite control, improving food intake control, and/or managing weight, the method comprising administering to an individual in need thereof or at risk thereof any of the compositions disclosed herein. The composition may be administered in an amount effective for increasing levels of Bifidobacterium in the individual. The composition may be administered to the individual in a unit dosage form, wherein the unit dosage form comprises an amount of the composition effective for increasing levels of Bifidobacterium in an individual.

Yet another aspect of the present disclosure is a method for one or more of reducing and/or preventing oxidative stress and/or treating and/or preventing age-associated diseases, the method comprising administering to an individual in need thereof or at risk thereof any of the compositions disclosed herein. The composition may be administered in an amount effective for increasing levels of 3-hydroxybutyrate in the individual. The composition may be administered to the individual in a unit dosage form, wherein the unit dosage form comprises an amount of the composition effective for increasing levels of 3-hydroxybutyrate in an individual.

Yet another aspect of the present disclosure is a method for improving lipid metabolism, the method comprising administering to an individual in need thereof or at risk thereof any of the compositions disclosed herein. The composition may be administered in an amount effective for increasing levels of 12,13-diHODE in the individual. The composition may be administered to the individual in a unit dosage form, wherein the unit dosage form comprises an amount of the composition effective for increasing levels of 12,13-diHODE in an individual.

The embodiments disclosed herein each include one or more advantageous features not provided by the compositions and methods of “Methods of Use of Oligosaccharide Compositions For Modulating Microbiota And Their Metabolic Products, And As Therapeutics For Health Application,” International Patent Publication No. WO2022241163, by Amicucci et al.; “Process For Preparing Enzymatically-Treated Food Compositions With Gos And Low Lactose Content And Food Compositions Thereof,” International Patent Publication No. WO2023057540, by Sabine et al.; “Milk Oligosaccharide Galactooligosaccharide Composition For Infant Formula Containing The Soluble Oligosaccharide Fraction Present In Milk, And Having A Low Level Of Monosaccharides, And A Process To Produce The Composition,” U.S. Patent Publication No. 20140087021, by Berrocal Rafael et al.; “Synthetic composition for treating metabolic disorders,” U.S. Pat. No. 11,529,364, by Elison et al.; “Immune system stimulating nutrition,” U.S. Pat. No. 10,124,016, by Potappel-Van'T Land Belinda et al.; “Milk products comprising high amounts of galactooligosaccharides (gos) and their production,” European Patent No. 3624594, by Tams J. W.; and “Enzymatic conversion of milk lactose to prebiotic galactooligosaccharides to produce low lactose yogurt,” by Raza, Iqbal, Ullah, Khan, and Imran in J Food Process Preserv. 2018; 42:e13586. https://doi.org/10.1111/jfpp.13586.

Additional features and advantages are described herein and will be apparent from the following Figures and Detailed Description.

### BRIEF DESCRIPTION OF THE DRAWINGS

FIG. 1 shows plasma 3-indole propionate levels after administration of the composition disclosed herein (IP) compared to administration of a control product (CP) of lactose-free skimmed milk.

FIG. 2 shows plasma 3-hydroxydecanoic acid levels after administration of the composition disclosed herein (IP) compared to administration of a control product (CP) of lactose-free skimmed milk.

FIG. 3 shows plasma 12,13-DiHODE levels after administration of the composition disclosed herein (IP) compared to administration of a control product (CP) of lactose-free skimmed milk.

FIG. 4 shows plasma indoxyl sulfate levels after administration of the composition disclosed herein (IP) compared to administration of a control product (CP) of lactose-free skimmed milk.

FIG. 5 shows plasma p-cresol sulfate levels after administration of the composition disclosed herein (IP) compared to administration of a control product (CP) of lactose-free skimmed milk.

FIG. 6 shows plasma acetate levels after administration of the composition disclosed herein (IP) compared to administration of a control product (CP) of lactose-free skimmed milk.

FIG. 7 shows plasma caprylic acid levels after administration of the composition disclosed herein (IP) compared to administration of a control product (CP) of lactose-free skimmed milk.

FIG. 8 shows plasma nicotinamide levels after administration of the composition disclosed herein (IP) compared to administration of a control product (CP) of lactose-free skimmed milk.

FIG. 9 shows plasma beta-alanine levels after administration of the composition disclosed herein (IP) compared to administration of a control product (CP) of lactose-free skimmed milk.

FIG. 10 shows plasma ketone body levels after administration of the composition disclosed herein (IP) compared to administration of a control product (CP) of lactose-free skimmed milk.

FIG. 11 shows fecal Bifidobacterium levels after administration of the composition disclosed herein (IP) compared to administration of a control product (CP) of lactose-free skimmed milk.

FIG. 12 shows fecal B. longum levels after administration of the composition disclosed herein (IP) compared to administration of a control product (CP) of lactose-free skimmed milk.

FIG. 13 shows fecal B. adolescentis levels after administration of the composition disclosed herein (IP) compared to administration of a control product (CP) of lactose-free skimmed milk.

FIG. 14 shows fecal B. bifidum levels after administration of the composition disclosed herein (IP) compared to administration of a control product (CP) of lactose-free skimmed milk.

FIG. 15 shows the Bifidobacterium shunt pathway abundance after administration of the composition disclosed herein (IP) compared to administration of a control product (CP) of lactose-free skimmed milk.

FIG. 16 shows the NAD salvage pathway abundance after administration of the composition disclosed herein (IP) compared to administration of a control product (CP) of lactose-free skimmed milk.

FIG. 17-22 show the increase of certain metabolites during fermentation of the composition of the present invention compared to GOS alone and a control.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a protein” or “the protein” includes two or more proteins.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions and methods disclosed herein may lack any element that is not specifically disclosed herein. Thus, a disclosure of an embodiment using the term “comprising” includes a disclosure of embodiments “consisting essentially of” and “consisting of” the components or steps identified.

The terms “at least one of” and “and/or” used respectively in the context of “at least one of X and Y” and “X and/or Y” should be interpreted as “X without Y,” or “Y without X,” or “both X and Y.” Where used herein, the terms “example” and “such as,” particularly when followed by a listing of terms, are merely exemplary and illustrative and should not be deemed to be exclusive or comprehensive.

As used herein, “related to,” “associated with” and “linked with” mean occurring concurrently, preferably mean caused by the same underlying condition, more preferably mean that one of the identified conditions is at least indirectly caused by the other identified condition, and most preferably mean that one of the identified conditions is directly caused by the other identified condition.

The term “chronic kidney disease” refers to a gradual loss of kidney function. Kidneys filter wastes and excess fluids from the blood, which are then removed in the urine. Advanced chronic kidney disease can cause dangerous levels of fluid, electrolytes and wastes to build up in the body. Chronic kidney disease occurs when a disease or condition impairs kidney function, causing kidney damage to worsen over several months or years. Diabetes and high blood pressure are two main risk factors.

The term “diabetes” shall refer to a disease that results in high glucose levels in the bloodstream.

The terms “food”, “food supplement”, “food product” and “food composition” mean a product or composition that is intended for ingestion by an individual such as a human and provides at least one nutrient to the individual. The compositions of the present disclosure, including the many embodiments described herein, can comprise, consist of, or consist essentially of the elements disclosed herein, as well as any additional or optional ingredients, components, or elements described herein or otherwise useful in a diet supplement.

“Prevention” includes reduction of risk, incidence and/or severity of a condition or disorder. The terms “treatment” and “treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

As used herein, the term “low lactose” shall refer to a composition with less than or equal to 30% of lactose.

As used herein, the term “low sugar” shall refer to a composition with less than or equal to 55% of sugar.

As used herein, a prophylactically or therapeutically “effective amount” is an amount that prevents a deficiency, treats a disease or medical condition in an individual, or, more generally, reduces symptoms, manages progression of the disease, or provides a nutritional, physiological, or medical benefit to the individual.

As used herein, the term “metabolic health” shall refer to the ability for your body to digest and absorb nutrients from food without abnormal or unhealthy spikes in blood sugar, blood lipid, inflammation, and/or insulin. “Improving metabolic health” shall refer to mechanisms for directly or indirectly reducing the incidence of spikes in blood sugar, inflammation, insulin, and/or blood fat. Additionally, “improving metabolic health” may refer to preventing and/or treating obesity, type II diabetes, heart disease, cognitive function, stroke, kidney disease, and/or fatty liver disease.

As used herein, the term “oxidative stress” refers to the state in an individual, or cell or tissue of an individual, of an imbalance between the production of reactive oxygen and the ability to detoxify the reactive intermediates or easily repair the resulting damage in a biological system. The natural reducing environment within cells is maintained by processes using a constant input of metabolic energy, and disturbances in this normal redox state can result in toxic effects through the production of, for example, free radicals and peroxides that damage cellular components, such as proteins, lipids, and/or DNA, for example.

The term “composition” may mean a food, beverage, dietary supplement, complete nutrition or oral nutritional supplement (ONS) or medical food composition, or mixture thereof.

The composition can be in solid form (e.g., powder) or in liquid form. The amount of the various ingredients can be expressed in g/100 g of the composition on a dry weight basis when it is in a solid form, e.g. a powder, or as a concentration in g/100 mL of the composition when it refers to a liquid form (this latter also encompasses liquid composition that may be obtained from a powder after reconstitution in a liquid such as water.)

The term “unit dosage form,” as used herein, refers to physically discrete units suitable as unitary dosages for human and animal subjects, each unit containing a predetermined quantity of the composition disclosed herein in an amount sufficient to produce the desired effect, in association with an acceptable diluent, carrier or vehicle. The specifications for the unit dosage form depend on the particular compounds employed, the effect to be achieved, and the pharmacodynamics associated with each compound in the host.

### EMBODIMENTS

In an aspect of the present disclosure, a method of improving metabolic health comprises administering to an individual in need thereof or at risk thereof a low sugar, low lactose, milk-based composition comprising galactooligosaccharide.

In some embodiments, the composition comprises 20-40% by dry weight of milk protein; and 32-55% by dry weight of a mixture of lactose, glucose, galactose and galactooligosaccharide.

In some embodiments, the composition comprises 35-55% by dry weight of a mixture of lactose, glucose, galactose and galactooligosaccharide.

In an embodiment, the mixture of lactose, glucose and galactose and galactooligosaccharide comprises: 0.1-30% by weight of lactose.

In an embodiment, the mixture of lactose, glucose and galactose and galactooligosaccharide comprises: 8-40% by weight of glucose.

In an embodiment, the mixture of lactose, glucose and galactose and galactooligosaccharide comprises: 0.1-20% by weight of galactose.

In an embodiment, the mixture of lactose, glucose and galactose and galactooligosaccharide comprises 40-90% by weight of galactooligosaccharide.

In an embodiment, the milk-based composition comprises: milk protein, preferably at 20-40% by dry weight of the composition; and a mixture of lactose, glucose, galactose and galactooligosaccharide, the mixture preferably at 32-55% by dry weight of the composition; preferably, the mixture comprising: 0.1-30% by weight of the lactose, 8-40% by weight of the glucose, 0.1-20% by weight of the galactose and 40-90% by weight of the galactooligosaccharide.

In another embodiments, the milk-based composition comprises:
milk protein, preferably at 20-40% by dry weight of the composition; and
a mixture of lactose, glucose, galactose and galactooligosaccharide, the mixture preferably at 35-55% by dry weight of the composition;
preferably, the mixture comprising: 0.1-30% by weight of the lactose, 8-40% by weight of the glucose, 0.1-20% by weight of the galactose and 40-90% by weight of the galactooligosaccharide.

Some embodiments provide a contribution from each of the components of the composition individually, in combination, and/or synergistically.

In certain embodiments of the invention, there are methods and compositions for improving metabolic health, the improvement of metabolic health caused directly or indirectly by increasing or decreasing levels of certain metabolites or certain bacteria such as Bifidobacteria in the individual. The individual may be of any age or state of health, although in particular embodiments the individual may be susceptible to particular medical conditions or physical states that are treated or prevented directly or indirectly with increased or decreased metabolite levels, or has a medical condition or physical state that are treated or prevented directly or indirectly with increased metabolite levels. The compositions delivered to the individual in such cases include at least a mixture of lactose, glucose, galactose and galactooligosaccharide, the mixture comprising: 0.1-30% by weight of lactose, 8-40% by weight of glucose, 0.1-20% by weight of galactose and 40-90% by weight of galactooligosaccharide, in particular to facilitate raising or lowering specific metabolite levels in the individual. One can measure specific metabolite levels through plasma or fecal samples, for example, in methods well known in the art.

In specific embodiments, improvement of metabolic health is associated with increased and/or decreased levels of certain metabolites, certain bacteria such as Bifidobacteria, or functional derivatives thereof. In particular embodiments, improving metabolic health comprises one or more of the following: (a) appetite or food intake control or regulation; (b) managing glucose levels; (c) weight management; (d) normalizing blood lipid profiles; (e) skin health; (f) reducing inflammation; (g) preventing and/or attenuating oxidative stress; (h) improved lipid/fat metabolism; (i) improving intestinal barrier; (j) reducing intestinal permeability; (k) modulation of glucose metabolism; (l) protection from fatty liver; (m) promoting growth of Faecalibacterium prausnitzii, (n) protecting kidneys, particularly in chronic kidney disease; (o) promoting growth of Bifidobacterium; (p) increasing Bifidobacterium shunt pathway; (q) increasing NAD salvage pathway (r) preservation of β-cell function; (s) preventing diabetes, (t) improving insulin sensitivity; (u) preventing and/or treating obesity; (x) regulating blood pressure; (y) maintain insulin response; and (z) combinations thereof.

The individual may be diagnosed with such condition(s) or may be suspected of having such condition(s) or may be susceptible to such condition(s). The individual may be treated with other therapy or therapies in addition to methods of the disclosure.

In one embodiment of the disclosure, there is a method of producing increased blood levels of certain metabolites, including 3-indole propionate, propionate, indoxyl-3-carboxylate, valerate, tricarballylic acid, imidazole acetate, acetate, N,N,-dimethylglycine, caprylic acid, nicotinamide, beta-alanine, ketone bodies, 3-hydroxybutyrate, 12,13-diHODE, and/or 3-hydroxydecanoic acid. Preferably, the composition is administered in an effective amount to increase one or more Bifidobacterium, including but not limited to B. longum, B. adolescentis, B. bifidum, in an individual in need thereof by administering the composition disclosed herein. The individual may be known to have a medical condition or physical state that would benefit from increased metabolite levels, or the individual may be suspected of having a medical condition or physical state that would benefit from increased metabolite levels, or increased beneficial fecal bacteria levels.

The method may comprise identifying the individual as having a medical condition or physical state that would benefit from increased metabolite levels, or increased beneficial fecal bacteria levels, and/or the method may comprise identifying the individual as at risk of a medical condition or physical state that would benefit from increased metabolite levels, or increased beneficial fecal bacteria levels. In particular embodiments, the individual subjected with methods and/or compositions of the invention is desiring prevention of one or more undesirable physical states (or the effects thereof, such as with aging) or medical conditions.

In specific embodiments, an individual is provided effective amounts of compositions as described herein for the explicit purpose of raising intracellular levels of 3-indole propionate, propionate, indoxyl-3-carboxylate, valerate, tricarballylic acid, imidazole acetate, acetate, N,N,-dimethylglycine, caprylic acid, nicotinamide, beta-alanine, ketone bodies, 3-hydroxybutyrate, 12,13-diHODE, and/or 3-hydroxydecanoic acid. Preferably, the composition is administered in an effective amount to increase one or more Bifidobacterium, including but not limited to B. longum, B. adolescentis, B. bifidum, because it is determined that the individual is afflicted with a condition for which such levels are directly or indirectly related. Additionally, an individual is provided effective amounts of compositions as described herein for the explicit purpose of decreasing levels of indoxyl sulfate and/or p-cresol sulfate because it is determined that the individual is afflicted with a condition for which such levels are directly or indirectly related.

In some embodiments, there is a method of preventing and/or treating pellagra, acne, skin cancer, and/or improving skin health in an individual. In some embodiments, there is a method of improving the level of blood lipids in individuals. In some embodiments, there is a method of improving cognition. In specific embodiments, administering the compositions contemplated herein increase levels of nicotinamide for which pellagra, skin cancer, acne, blood lipid profile, improved cognition, and/or skin health are directly or indirectly related.

In some embodiments, there is a method of preventing and/or treating obesity in an individual. For example, the method of preventing and/or treating obesity may include improving appetite control, improving food intake control, managing weight, and managing glucose, such as through insulin sensitivity. In some embodiments, there is a method of preventing and/or treating viral respiratory infections, such as respiratory syncytial virus (RSV). In some embodiments, there is a method of treating and/or preventing cognitive decline. In specific embodiments, administering the compositions contemplated herein increase levels of acetate for which obesity, cognitive decline, and viral respiratory infections are directly or indirectly related.

In some embodiments, there is a method of preventing and/or treating obesity in an individual. For example, the method of preventing and/or treating obesity may include improving appetite control, improving food intake control, managing weight, and managing glucose, such as through insulin sensitivity. In some embodiment, there is a method of preventing and/or treating viral respiratory infections, such as respiratory syncytial virus (RSV). In some embodiments, there is a method of protecting an individual against pathogens. In specific embodiments, administering the compositions contemplated herein increase levels of Bifidobacterium, such as adolescentis, bifidum, and/or longum, for which obesity, viral respiratory infections, and/or protection from pathogens are directly or indirectly related.

In some embodiments, there is a method of treating or preventing chronic kidney disease or protecting kidney function. In specific embodiments, administering the compositions contemplated herein increase levels of 3-indole propionate and decrease levels of p-cresol sulfate and indoxyl sulfate for which chronic kidney disease and kidney function are directly or indirectly related.

In some embodiments, there is a method of reducing and/or preventing oxidative stress in an individual. In a specific embodiment, the methods are useful for treating and/or preventing medical conditions associated with oxidative stress. In particular cases, the present invention provides methods and compositions useful for aging. In specific embodiments, administering the compositions contemplated herein increase levels of 3-Hydroxybutyrate for which oxidative stress and age-associated diseases are directly or indirectly related.

In some embodiments, there is a method of improving lipid metabolism in an individual. In specific embodiments, administering the compositions contemplated herein increase levels of 12,13-diHODE for which lipid metabolism is directly or indirectly related.

In some embodiments, the composition comprises 20-40% by dry weight of milk protein. Milk protein includes any protein or combination of proteins derived from milk. The milk proteins may have been derived from milk in any suitable way. In a preferred embodiment, the composition comprises 25-40%, more preferably 30-40% by dry weight of milk protein.

In some embodiments, the food ingredient further comprises 35-55% or 32%-55% by dry weight of a mixture of lactose, glucose, galactose and GOS, the mixture comprising: 0.1-30% by weight of lactose, 8-40% by weight of glucose, 0.1-20% by weight of galactose and 40-90% by weight of GOS. In a preferred embodiment, the food ingredient comprises 45-55% by dry weight of the mixture of lactose, glucose, galactose and GOS; in a more preferred embodiment, the food ingredient comprises 50-55% by dry weight of the mixture of lactose, glucose, galactose and GOS. In another preferred embodiment of the invention, the mixture of lactose, glucose, galactose and GOS comprises 60-90% by weight of GOS, more preferred 70-90% by weight of GOS. In yet another preferred embodiment, the mixture of lactose, glucose, galactose and GOS contains: 1-20% by weight of lactose, 15-35% by weight of glucose, 1-10% by weight of galactose and 50-85% by weight of GOS. In a more preferred embodiment of the invention, the mixture of lactose, glucose, galactose and GOS contains: 1-14% by weight of lactose, 15-29% by weight of glucose, 1-8% by weight of galactose and 70-90% by weight of GOS.

The composition may further comprise 0.5-30% by dry weight of milk fat and/or minerals derived from milk, preferably 2-20%, more preferably 6-10% by dry weight of milk fat and/or minerals derived from milk. Milk fat includes fat derived from milk in any suitable way. Milk derived minerals include minerals derived from milk in any suitable way, e.g. calcium, sodium, potassium, phosphorus and/or magnesium salts. Milk fat and/or milk derived minerals may originate from liquid milk, such as skim milk, partially skimmed milk and/or whole milk, or from milk powder, such as e.g. skim milk powder and/or whole milk powder.

In some embodiments, the mixture of lactose, glucose, galactose and GOS comprises 0.1-30%, such as 0.1-5%, 5-10%, 10-15%, 15-20%, 20-25%, or 25-30% by weight of lactose. In some embodiments, the mixture of lactose, glucose, galactose and GOS comprises 8-40%, such as 8-16%, 16-24%, 24-32%, or 32-40%, by weight of glucose. In some embodiments, the mixture of lactose, glucose, galactose and GOS comprises 0.1-20%, such as 0.1-5%, 5-10%, 10-15%, or 15-20%, by weight of galactose. In some embodiments, the mixture of lactose, glucose, galactose and GOS comprises 40-90%, such as 40-50%, 50-60%, 60-70%, or 70-80% by weight of GOS.

In a preferred embodiment, the composition further comprises 5-20% by dry weight of vegetable oil, more preferably 8-15% by dry weight of vegetable oil. The vegetable oil may be any suitable vegetable oil, preferably palm oil. In one embodiment, the composition relates to a cocoa, coffee, fruit based and/or malt beverage powder comprising: 10-35% by weight of non-fat milk solids; 0-65% by weight of malt extract solids; 0-15% by weight of cocoa solids; 6-15% by weight of vegetable oil and/or milk fat; 0-25% by weight of sucrose; 1-6% by weight of glucose; 0.1-5% by weight of lactose; and 3-10% by weight of GOS.

Non-fat milk solids may be derived from any suitable milk source, such as e.g. liquid milk, e.g. skim milk and/or whole milk, and/or milk powder, e.g. skim milk powder and/or whole milk powder. If liquid milk is used, it may be concentrated, e.g. by evaporation or filtration. Malt extract solids may be in any suitable form, e.g. in the form of a powdered or liquid extract of malted barley and/or wheat. Cocoa solids may be from any suitable cocoa source, e.g. in the form of cocoa powder, cocoa mass and/or cocoa butter. Vegetable oil may be any suitable vegetable oil, preferably palm oil. Milk fat may be derived from any suitable milk source, such as e.g. liquid milk or cream, e.g. skim milk, cream, and/or whole milk, and/or milk powder, e.g. skim milk powder, cream powder, and/or whole milk powder, and/or may be in the form of butter, butter oil and/or anhydrous milk fat. If liquid milk or cream is used, it may be concentrated, e.g. by evaporation or filtration.

Glucose, lactose and GOS are preferably derived from the composition of the present invention. A cocoa and/or malt beverage powder of the invention preferably comprises 10-30% by dry weight of the composition of the invention, more preferably 15-25% by dry weight of the composition of the invention. In a preferred embodiment of the invention, the cocoa and/or malt beverage powder comprises 24% by weight of glucose; 0.1-2% by weight of lactose; and 5-10% by weight of GOS.

A cocoa and/or malt beverage powder of the invention may comprise sucrose. Due to the amounts of lactose, glucose and GOS present in the product, the amount of sucrose may be reduced as compared to a conventional cocoa and/or malt beverage powder while retaining an acceptable taste, sweetness and texture. In a preferred embodiment, a cocoa and/or malt beverage product of the invention does not comprise sucrose. In another preferred embodiment, a cocoa and/or malt beverage powder of the invention comprises 1-25% by weight of sucrose, more preferably 5-20% by weight of sucrose.

A cocoa and/or malt beverage powder of the invention may comprise any other suitable ingredients known in the art, such as e.g. vitamins, minerals, buffer salts, emulsifiers and stabilizers.

A cocoa and/or malt beverage powder of the invention may be produced by any suitable method known in the art. The ingredients may e.g. be in powder form and mixed in the dry state, some or all ingredients may be mixed in aqueous solution/suspension and subsequently dried to a powder, or some or all powdered ingredients may e.g. be co-agglomerated to produce a powder with improved solubility.

In a preferred embodiment, the invention relates to a cocoa and malt beverage powder comprising 15-35% by weight of non-fat milk solids; 20-50% by weight of malt extract solids; 5-15% by weight of cocoa solids; 6-15% by weight of vegetable oil and/or milk fat; 0-25% by weight of sucrose; 1-6% by weight of glucose; 0.1-5% by weight of lactose; and 3-10% by weight of galactooligosaccharide.

In one embodiment, the composition relates to a liquid cocoa and/or malt beverage product comprising: 3-10% by weight of non-fat milk solids; 1-6% by weight of malt extract solids; 0-5% by weight of cocoa solids; 0.3-5% by weight of vegetable oil and/or milk fat; 0-5% by weight of sucrose; 0.4-1.5% by weight of glucose; 0.01-1% by weight of lactose; and 0.5-5% by weight of GOS.

A liquid cocoa and/or malt beverage product is a cocoa and/or malt beverage product in liquid form which is suitable to be consumed without addition of liquid. It may e.g. be packed in a container from which it can be consumed directly upon opening.

Non-fat milk solids may be derived from any suitable milk source, such as e.g. liquid milk, e.g. skim milk and/or whole milk, and/or milk powder, e.g. skim milk powder and/or whole milk powder. If liquid milk is used, it may be concentrated, e.g. by evaporation or filtration. Malt extract solids may be in any suitable form, e.g. in the form of a powdered or liquid extract of malted barley and/or wheat. Cocoa solids may be from any suitable cocoa source, e.g. in the form of cocoa powder, cocoa mass and/or cocoa butter. Vegetable oil may be any suitable vegetable oil, preferably palm oil. Milk fat may be derived from any suitable milk source, such as e.g. liquid milk or cream, e.g. skim milk, cream, and/or whole milk, and/or milk powder, e.g. skim milk powder, cream powder, and/or whole milk powder, and/or may be in the form of butter, butter oil and/or anhydrous milk fat. If liquid milk or cream is used, it may be concentrated, e.g. by evaporation or filtration.

Glucose, lactose and GOS are preferably derived from the composition of the present invention. A liquid cocoa and/or malt beverage product of the invention preferably comprises 10-30% by dry weight of the composition of the invention, more preferably 15-25% by dry weight. In a preferred embodiment, the liquid cocoa and/or malt beverage product comprises 0.5-1% by weight of glucose; 0.01-0.5% by weight of lactose; and 1-4% by weight of GOS.

A liquid cocoa and/or malt beverage product of the invention may comprise sucrose. Due to the amounts of lactose, glucose and GOS present in the product, the amount of sucrose may be reduced as compared to a conventional cocoa and/or malt beverage powder while retaining an acceptable taste, sweetness and texture. In a preferred embodiment, a cocoa and/or malt beverage product of the invention does not comprise sucrose. In another preferred embodiment, a cocoa and/or malt beverage powder of the invention comprises 0.1-5% by weight of sucrose, more preferably 1-3% by weight of sucrose.

A liquid cocoa and/or malt beverage product of the invention may comprise any other suitable ingredients known in the art, such as e.g. vitamins, minerals, buffer salts, emulsifiers and stabilizers.

A liquid cocoa and/or malt beverage product of the invention may be produced in any suitable way known in the art. The ingredients may be in liquid and/or powdered form and may be mixed into an aqueous solution/suspension, e.g. with the addition of water to achieve the desired concentration of ingredients. A food ingredient of the invention is preferably added in liquid form, but may also be added in powdered form.

In a preferred embodiment, the composition relates to a liquid cocoa and/or malt beverage product comprising: 3-10% by weight of non-fat milk solids; 1-6% by weight of malt extract solids; 1-5% by weight of cocoa solids; 0.3-5% by weight of vegetable oil and/or milk fat; 0-5% by weight of sucrose; 0.4-1.5% by weight of glucose; 0.01-1% by weight of lactose; and 0.5-5% by weight of GOS.

The composition can be in any oral nutritional form, e.g., as a health drink, as a ready-made drink, optionally as a soft drink, including juices, coffee, tea, milk-shake, yogurt drink, smoothie or soy-based drink; in a food bar; a powder, such as in sachet form; or dispersed in foods of any sort, such as baked products, cereal bars, dairy bars, snack-foods, soups, breakfast cereals, muesli, candies, tabs, cookies, biscuits, crackers (such as rice crackers), and dairy and plant-based products.

In some embodiments, the composition relates to a ready to drink beverage. The % weight are based on dry matter of the composition before reconstitution. In an embodiment, the ready to drink beverage comprises 32%-55% of a mixture of Lactose, Glucose, Galactose, and GOS. In an embodiment, the composition comprises 40-45% by dry weight of the mixture of lactose, glucose, galactose and GOS. In an embodiment, the composition comprises 45-50% by dry weight of the mixture of lactose, glucose, galactose and GOS. In an embodiment, the food ingredient comprises 32-37% by dry weight of the mixture of lactose, glucose, galactose and GOS.

In some embodiments, the mixture of Lactose, Glucose, Galactose and GOS in the composition contains 1-25% by weight of lactose, such as 1-5%, 5-10%, 10-15%, 15-20%, or 20-25% by weight of lactose. In some embodiments, the mixture of Lactose, Glucose, Galactose and GOS in the composition contains 15-35% by weight of glucose, such as 15-20%, 20-25%, 25-30%, or 30-35% by weight of glucose. In some embodiments, the mixture of Lactose, Glucose, Galactose and GOS in the composition contains 1-10% by weight of galactose, such as 1-2%, 2-4%, 4-6%, 6-8%, or 8-10% by weight of galactose. In some embodiments, the mixture of Lactose, Glucose, Galactose and GOS in the composition contains 50-85% by weight of GOS, such as 50-60%, 60-70%, 70-80%, or 80-85% by weight of GOS.

In a preferred embodiment, the composition comprises 40-45% of a mixture of Lactose, Glucose, Galactose and GOS. In a preferred embodiment, the mixture of Lactose, Glucose, Galactose and GOS in the composition contains 10-20% by weight of lactose. In a preferred embodiment, the mixture of Lactose, Glucose, Galactose and GOS in the composition contains 15-25% by weight of glucose. In a preferred embodiment, the mixture of Lactose, Glucose, Galactose and GOS in the composition contains 0.1-5% by weight of galactose. In a preferred embodiment, the mixture of Lactose, Glucose, Galactose and GOS in the composition contains 57-70% by weight of GOS.

In some embodiments, the ready to drink beverage includes milk. In some embodiments, the milk includes full milk, skim milk, and other types of milk. In some embodiments, the ready to drink beverage is optionally fortified with one or more vitamins and minerals. In a preferred embodiment, the one or more vitamins include vitamins for providing a benefit, such as improving immunity and/or mobility.

The composition can be administered to the individual at least two days per week, at least three days per week, all seven days of the week respecting serving size; for at least one week, at least one month, at least two months, at least three months, at least six months, or even longer. In some embodiments, the composition is administered to the individual consecutively for a number of days. In an embodiment, the composition can be administered to the individual daily for at least 30, 60 or 90 consecutive days.

In some embodiments, the serving size of the composition is an amount effective to increase certain metabolites or certain bacteria such as Bifidobacteria, for example, about 100 ml, about 200 ml, or about 300 ml of the composition. In some embodiments, the composition is administered in a unit dosage form that comprises an amount of the composition effective for increasing or decreasing certain metabolites and improving metabolic health.

In view of the disclosures herein, an embodiment is a method for improving metabolic health, the method comprising administering to an individual in need thereof a low sugar, low lactose, milk-based composition comprising galactooligosaccharide. In some embodiments, the composition comprises milk protein, preferably at 20-40% by dry weight of the composition; and a mixture of lactose, glucose, galactose and galactooligosaccharide, the mixture preferably at 35-55%, most preferably 32-55%, by dry weight of the composition; preferably, the mixture comprising: 0.1-30% by weight of the lactose, 8-40% by weight of the glucose, 0.1-20% by weight of the galactose and 40-90% by weight of the galactooligosaccharide.

The improving of the metabolic health can comprise at least one result selected from the group consisting of: (a) appetite or food intake control or regulation; (b) managing glucose levels; (c) weight management; (d) normalizing blood lipid profiles; (e) skin health; (f) reducing inflammation; (g) preventing and/or attenuating oxidative stress; (h) improved lipid/fat metabolism; (i) improving intestinal barrier; (j) reducing intestinal permeability; (k) modulation of glucose metabolism; (l) protection from fatty liver; (m) promoting growth of Faecalibacterium prausnitzii, (n) protecting kidneys, particularly in chronic kidney disease; (o) promoting growth of Bifidobacterium; (p) increasing Bifidobacterium shunt pathway; (q) increasing NAD salvage pathway; (r) preservation of 0-cell function; (s) preventing diabetes, (t) improving insulin sensitivity; (u) preventing and/or treating obesity; (v) regulating blood pressure; (w) maintain insulin response; (x) preventing and/or reducing affects of parasites; and (y) combinations thereof.

The composition may further comprise 0.5-30% by dry weight of at least one of milk fat or mineral derived from milk, preferably a combination thereof. The composition may further comprise 5-20% by dry weight of vegetable oil.

Preferably the composition is administered in an amount effective to increase one or more metabolite levels selected from the group consisting of: 3-indole propionate, propionate, indole-3-carboxylate, valerate, tricarballylic acid, imidazole acetate, N,N,-dimethylglycine, caprylic acid, nicotinamide, beta-alanine, ketone bodies, 3-hydroxybutyrate, 12,13-diHODE, and/or 3-hydroxydecanoic acid. Preferably, the composition is administered in an effective amount to increase one or more Bifidobacterium, including but not limited to B. longum, B. adolescentis, B. bifidum. Preferably, the composition is administered in an effective amount to increase one or more Bifidobacterium, including but not limited to B. longum, B. adolescentis, B. bifidum.

Another aspect of the present disclosure is a method for one or more of preventing oxidative stress, preventing and/or treating type II diabetes, reducing inflammation, improving the intestinal barrier, reducing intestinal permeability, modulating glucose metabolism, improving insulin sensitivity, preventing obesity, appetite regulation, and/or protecting against fatty liver, the method comprising administering to an individual in need thereof or at risk thereof any of the compositions disclosed herein. The composition may be administered in an amount effective for increasing levels of 3-indole propionate or propionate in the individual. The composition may be administered to the individual in a unit dosage form, wherein the unit dosage form comprises an amount of the composition effective for increasing levels of 3-indole propionate or propionate in an individual. Research has shown the increasing propionate levels has been linked to the listed benefits (See Vinolo M A, Rodrigues H G, Nachbar R T, Curi R. Regulation of inflammation by short chain fatty acids. Nutrients. 2011; 3:858-76: https://www.mdpi.com/2072-6643/3/10/858; De Vadder, F. et al. Microbiota-generated metabolites promote metabolic benefits via gut-brain neural circuits. Cell 156, 84-96 (2014); Chambers, E. S. et al. Effects of targeted delivery of propionate to the human colon on appetite regulation, body weight maintenance and adiposity in overweight adults. Gut 64, 1744-1754 (2015).; Xiong, Y. et al. Short-chain fatty acids stimulate leptin production in adipocytes through the G protein coupled receptor GPR41. Proc. Natl Acad. Sci. USA 101, 1045-1050 (2004).; Goswami, C., Iwasaki, Y. & Yada, T. Short-chain fatty acids suppress food intake by activating vagal afferent neurons. J. Nutr. Biochem. 57, 130-135 (2018).; Mills, S. W., Montgomery, S. H., & Morck, D. W. (2006). Evaluation of the effects of short-chain fatty acids and extracellular pH on bovine neutrophil function in vitro. American Journal of Veterinary Research, 67(11), 1901-1907.; Pingitore A, Chambers E S, Hill T, Maldonado I R, Liu B, Bewick G, Morrison D J, Preston T, Wallis G A, Tedford C, Castanera Gonzilez R, Huang G C, Choudhary P, Frost G and Persaud S J. The diet-derived short chain fatty acid propionate improves beta-cell function in humans and stimulates insulin secretion from human islets in vitro, Diabetes, Obesity and Metabolism 2017; 19(2): 257-265.; Chambers E S, Byrne C S, Aspey K, Chen Y, Khan S, Morrison D J, Frost G. Acute oral sodium propionate supplementation raises resting energy expenditure and lipid oxidation in fasted humans. Diabetes Obes Metab. 2018 April; 20(4):1034-1039. doi: 10.1111/dom.13159. Epub 2017 Dec. 17. PMID: 29134744; PMCID: PMC5873405.; Chambers E S, Viardot A, Psichas A, Morrison D J, Murphy K G, Zac-Varghese S E, MacDougall K, Preston T, Tedford C, Finlayson G S, Blundell J E, Bell J D, Thomas E L, Mt-Isa S, Ashby D, Gibson G R, Kolida S, Dhillo W S, Bloom S R, Morley W, Clegg S, Frost G. Effects of targeted delivery of propionate to the human colon on appetite regulation, body weight maintenance and adiposity in overweight adults. Gut. 2015 November; 64(11):1744-54. doi: 10.1136/gutjnl-2014-307913. Epub 2014 Dec. 10. PMID: 25500202; PMCID: PMC4680171.; Chambers E S, Viardot A, Psichas A, Morrison D J, Murphy K G, Zac-Varghese S E, MacDougall K, Preston T, Tedford C, Finlayson G S, Blundell J E, Bell J D, Thomas E L, Mt-Isa S, Ashby D, Gibson G R, Kolida S, Dhillo W S, Bloom S R, Morley W, Clegg S, Frost G. Effects of targeted delivery of propionate to the human colon on appetite regulation, body weight maintenance and adiposity in overweight adults. Gut. 2015 November; 64(11):1744-54. doi: 10.1136/gutjnl-2014-307913. Epub 2014 Dec. 10. PMID: 25500202; PMCID: PMC4680171., the entireties of each of which are incorporated herein by reference.)

Yet another aspect of the present disclosure is a method for preventing and/or treating kidney disease, the method comprising administering to an individual in need thereof or at risk thereof any of the compositions disclosed herein. The composition may be administered in an amount effective for increasing levels of 3-indole propionate in an individual and decreased levels of at least one metabolite selected from the group consisting of p-cresol sulfate and indoxyl sulfate. The composition may be administered to the individual in a unit dosage form, wherein the unit dosage form comprises an amount of the composition effective for increasing levels of 3-indole propionate and decreasing levels of at least one of metabolite selected from the group consisting of p-cresol sulfate and indoxyl sulfate in the individual.

Yet another aspect of the present disclosure is a method for one or more of treating or preventing pellagra, acne, or skin cancer, improving skin health, and/or improving the level of blood lipids, the method comprising administering to an individual in need thereof or at risk thereof any of the compositions disclosed herein. The composition may be administered in an amount effective for increasing levels of nicotinamide in the individual. The composition may be administered to the individual in a unit dosage form, wherein the unit dosage form comprises an amount of the composition effective for increasing levels of nicotinamide in an individual.

Yet another aspect of the present disclosure is a method for one or more of preventing and/or treating obesity, improving appetite control, improving food intake control, and/or managing weight, the method comprising administering to an individual in need thereof or at risk thereof any of the compositions disclosed herein. The composition may be administered in an amount effective for increasing levels of acetate in the individual. The composition may be administered to the individual in a unit dosage form, wherein the unit dosage form comprises an amount of the composition effective for increasing levels of acetate in an individual.

Yet another aspect of the present disclosure is a method for one or more of preventing and/or treating obesity, improving appetite control, improving food intake control, and/or managing weight, the method comprising administering to an individual in need thereof or at risk thereof any of the compositions disclosed herein. The composition may be administered in an amount effective for increasing levels of Bifidobacterium in the individual. The composition may be administered to the individual in a unit dosage form, wherein the unit dosage form comprises an amount of the composition effective for increasing levels of Bifidobacterium in an individual.

Yet another aspect of the present disclosure is a method for one or more of reducing and/or preventing oxidative stress and/or treating and/or preventing age-associated diseases, the method comprising administering to an individual in need thereof or at risk thereof any of the compositions disclosed herein. The composition may be administered in an amount effective for increasing levels of 3-hydroxybutyrate in the individual. The composition may be administered to the individual in a unit dosage form, wherein the unit dosage form comprises an amount of the composition effective for increasing levels of 3-hydroxybutyrate in an individual.

Yet another aspect of the present disclosure is a method for improving lipid/fat metabolism, the method comprising administering to an individual in need thereof or at risk thereof any of the compositions disclosed herein. The composition may be administered in an amount effective for increasing levels of 12,13-diHODE in the individual. The composition may be administered to the individual in a unit dosage form, wherein the unit dosage form comprises an amount of the composition effective for increasing levels of 12,13-diHODE in an individual.

Yet another aspect of the present disclosure is a method for reducing inflammation, and modulating glucose metabolism, the method comprising administering to an individual in need thereof or at risk thereof any of the compositions disclosed herein. The composition may be administered in an amount effective for increasing levels of valerate in the individual. The composition may be administered to the individual in a unit dosage form, wherein the unit dosage form comprises an amount of the composition effective for increasing levels of valerate in an individual. Research has shown increasing levels of valerate has been linked to the listed benefits (See Zhu L, Li G, Liang Z, Qi T, Deng K, Yu J, Peng Y, Zheng J, Song Y, Chang X. Microbiota-assisted iron uptake promotes immune tolerance in the intestine. Nat Commun. 2023 May 15; 14(1):2790. doi: 10.1038/s41467-023-38444-2. PMID: 37188703; PMCID: PMC10185671.; Luu M., Pautz S., Kohl V., Singh R., Romero R., Lucas S., Hofmann J., Raifer H., Vachharajani N., Carrascosa L. C., et al. The Short-Chain Fatty Acid Pentanoate Suppresses Autoimmunity by Modulating the Metabolic-Epigenetic Crosstalk in Lymphocytes. Nat. Commun. 2019; 10:760. doi: 10.1038/s41467-019-08711-2; Gao, G., Zhou, J., Wang, H. et al. Effects of valerate on intestinal barrier function in cultured Caco-2 epithelial cell monolayers. Mol Biol Rep 49, 1817-1825 (2022). https://doi.org/10.1007/s11033-021-06991-w; Han J-H, Kim I-S, Jung S-H, Lee S-G, Son H-Y, Myung C-S (2014) The Effects of Propionate and Valerate on Insulin Responsiveness for Glucose Uptake in 3T3-L1 Adipocytes and C2C12 Myotubes via G Protein-Coupled Receptor 41. PLoS ONE 9(4): e95268. https://doi.org/10.1371/journal.pone.0095268 the entireties of each of which are incorporated herein by reference.)

Yet another aspect of the present disclosure is a method for reducing inflammation, the method comprising administering to an individual in need thereof or at risk thereof any of the compositions disclosed herein. The composition may be administered in an amount effective for increasing levels of Tricarballylic acid in the individual. The composition may be administered to the individual in a unit dosage form, wherein the unit dosage form comprises an amount of the composition effective for increasing levels of Tricarballylic acid in an individual. Research has shown increasing levels of Tricarballylic acid has been linked to the listed benefits (See Ashbrook M J, McDonough K L, Pituch J J, Christopherson P L, Cornell T T, Selewski D T, Shanley T P, Blatt N B. Citrate modulates lipopolysaccharide-induced monocyte inflammatory responses. Clin Exp Immunol. 2015 June; 180(3):520-30. doi: 10.1111/cei., the entirety of which is incorporated herein by reference.)

Yet another aspect of the present disclosure is a method for regulating blood pressure, the method comprising administering to an individual in need thereof or at risk thereof any of the compositions disclosed herein. The composition may be administered in an amount effective for increasing levels of imidazole acetate in the individual. The composition may be administered to the individual in a unit dosage form, wherein the unit dosage form comprises an amount of the composition effective for increasing levels of imidazole acetate in an individual. Research has shown increasing levels of imidazole acetate has been linked to the listed benefits (See Tunnicliff 1998, Pharmacology and Function of Imidazole 4-Acetic Acid in Brain. Gen Pharmacol. 1998 October; 31(4):503-9. doi: 10.1016/s0306-3623(98)00079-2., the entirety of which is incorporated herein by reference.)

Yet another aspect of the present disclosure is a method for improving the intestinal barrier, maintaining normal insulin response, and reducing oxidative stress, the method comprising administering to an individual in need thereof or at risk thereof any of the compositions disclosed herein. The composition may be administered in an amount effective for increasing levels of N,N-Dimethylglycine in the individual. The composition may be administered to the individual in a unit dosage form, wherein the unit dosage form comprises an amount of the composition effective for increasing levels of N,N-Dimethylglycine in an individual. Research has shown increasing levels of N,N-Dimethylglycine has been linked to the listed benefits (See Wang et al. 2022. Heat stress-induced intestinal barrier damage and dimethylglycine alleviates via improving the metabolism function of microbiota gut brain axis. Ecotoxicology and Environmental Safety Volume 244, 1 Oct. 2022, 114053. https://www.sciencedirect.com/science/article/pii/S0147651322008934; Magnusson et al. 2015. Dimethylglycine Deficiency and the Development of Diabetes. Diabetes. 2015 August; 64(8):3010-6. doi: 10.2337/db14-1863. Epub 2015 Mar. 20.; Takahashi et al. 2016. N, N-Dimethylglycine decreases oxidative stress and improves in vitro development of bovine embryos. J Reprod Dev. 2016 April; 62(2): 209-212. doi: 10.1262/jrd.2015-149, the entireties of each of which are incorporated herein by reference.)

### EXAMPLES

### Example 1

The following non-limiting example generally illustrates the concepts underlying the embodiments disclosed herein.

The inventors examined the effect of administering the composition disclosed herein, versus lactose-free milk, in 26 healthy adults. Table 1 shows the composition administered versus the control.

TABLE 1
Active composition versus control composition
administered to individuals

| | |
|---|---|
| Control | Active Composition |

| | | | | |
|---|---|---|---|---|
| GOS | 0 | g | 9.2 | g |
| Sugar | 15 | g | 6.0 | g |
| Lactose | <0.1 | g | 1.8 | g |
| Fat | <0.5 | g | <0.5 | g |
| Protein | 10.2 | g | 10.2 | g |
| Calories | 105 | kcal | 111 | kcal |

Specifically, the inventors employed targeted and untargeted metabolomics for plasma samples and metagenomics for fecal samples. The composition disclosed herein was administered once daily for 2 weeks in a 200 ml drink portion.

92 plasma and fecal samples were analyzed using liquid chromatography high resolution mass spectrometer. The features (characterized by their retention time and accurate m z) were linearized and normalized. The features were annotated by their exact mass (m z) according to the Human Metabolome database (HMDB). Different statistical approaches were tested to study the effect of the administration of the composition. A statistical analysis was performed with linear models (after log transformation of the data) considering the period and the treatment to study the effect of the administration of the composition. Statistical significance was analyzed with a type 2 analysis of covariance and a false discovery rate estimate was performed. p<0.05 was considered as a significant change.

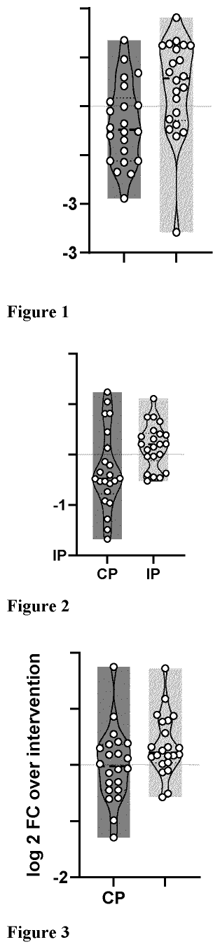

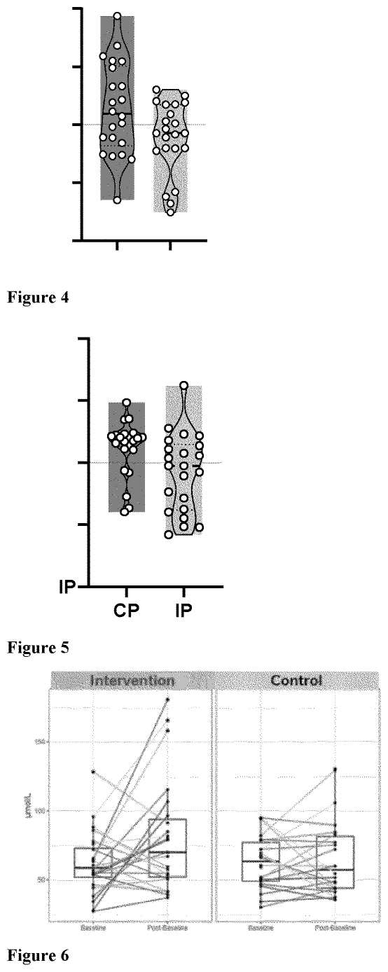

Among the significantly annotated features reported in plasma, the inventors found an increase for 3-indole propionic acid (FC of 0.77, p=0.01, with its identity confirmed by injecting a pure standard) as shown in FIG. 1. Two additional features, which were putatively annotated as 3-hydroxydecanoic acid and 12,13-DiHODE (a lineolic acid derivative), were also significantly increased with the intervention of the current composition (FC<0.5). FIG. 2 and FIG. 3 show the increases levels of 3-hydroxydecanoic acid and 12,13-DiHODE, respectively, of the intervention group compared to the control group. As seen in FIGS. 4 and 5, the two uremic toxins indoxyl-sulfate and p-cresol sulfate were significantly (p<0.05) reduced in the intervention group with a FC of −0.52 and −0.83, respectively.

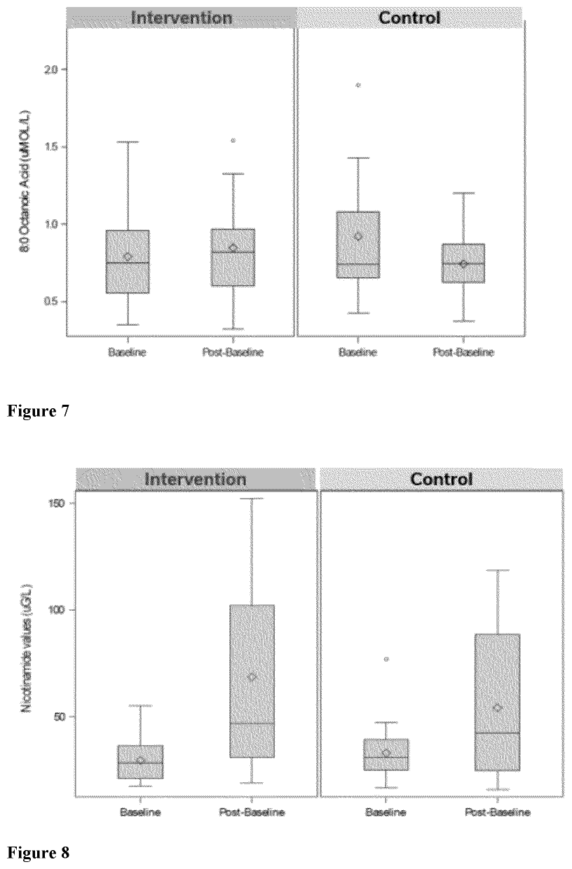

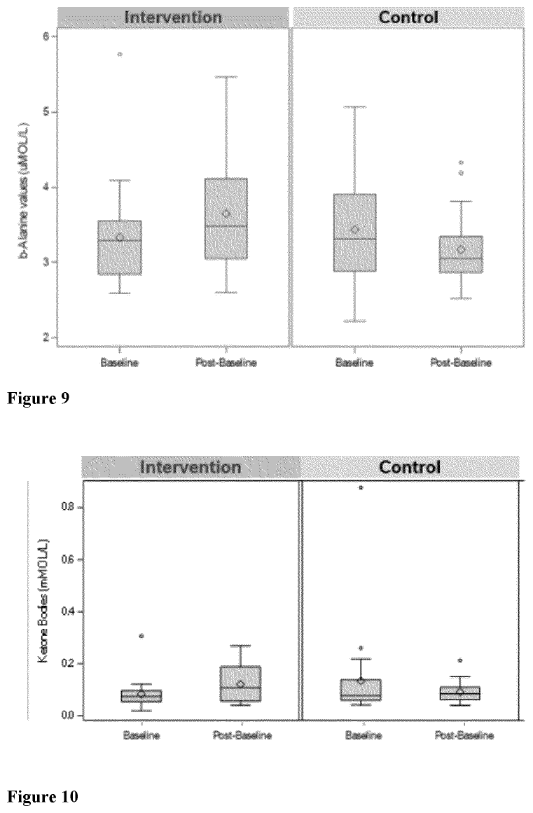

The results of the targeted metabolomics for plasma samples are shown in FIGS. 6 through 10. Plasma levels of acetate, caprylic acid, nicotinamide, and beta-alanine were significantly increased in the intervention group as shown in FIGS. 6, 7, 8, and 9, respectively. Additionally, the level of ketone bodies showed an increase in the intervention group as shown in FIG. 10, but no dietary intake information was obtained on the study.

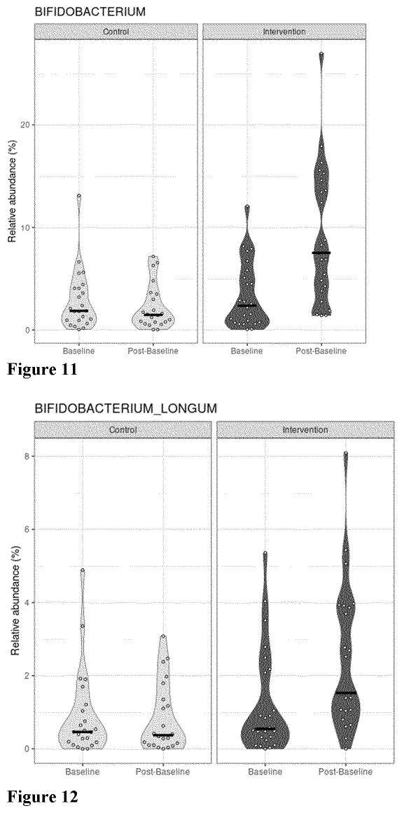

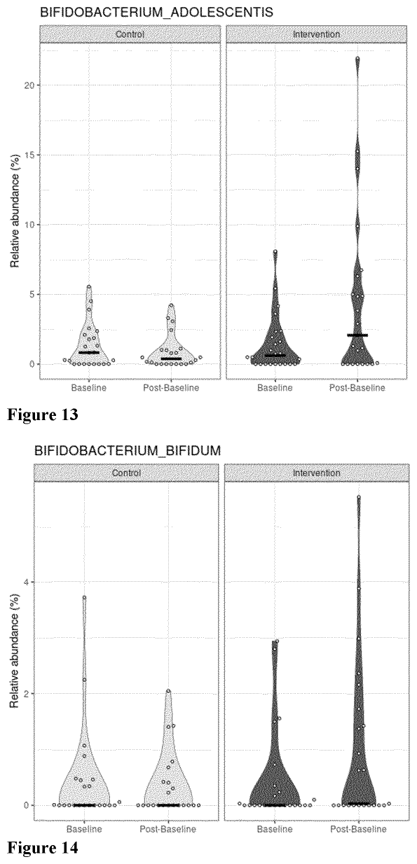

The results of the metagenomics for fecal samples are shown in FIGS. 11 through 14. Bifidobacterium levels significantly increased in the intervention group as shown in FIG. 11. Specifically, fecal levels of B. longum, B. adolescentis, and B. bifidum significantly increased in the intervention group as seen in FIGS. 12, 13, and 14, respectively.

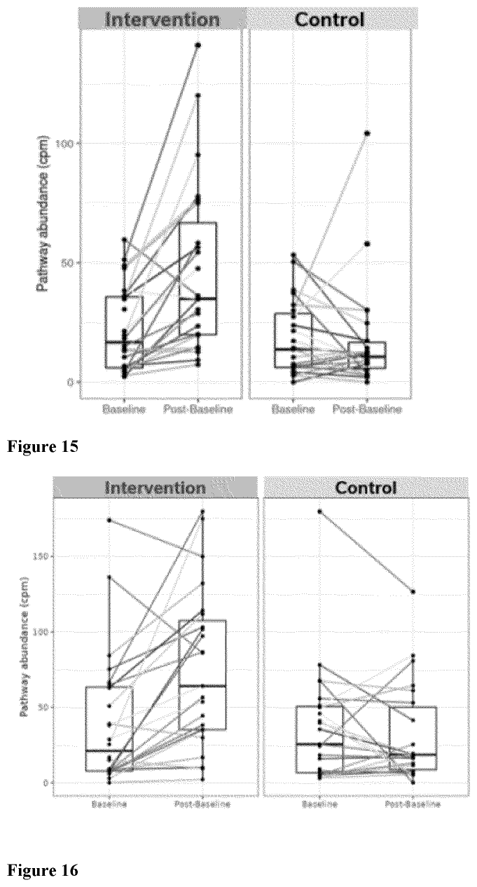

FIGS. 15 and 16 show the effect of administration on the Bifidobacterium shunt pathways and NAD salvage pathways.

An overview of targeted and untargeted metabolomics and metagenomics results is shown in the Table 2.

TABLE 2
Metabolite levels after administration of
the composition of the present disclosure

| Metabolite | Change | P-value |
|---|---|---|
| Acetate | + | 0.021 |
| Caprylic Acid | + | 0.0252 |
| Nicotinamide | + | 0.0245 |
| Beta-Alanine | + | 0.0077 |
| Bifidobacterium | + | <0.0001 |
| B. longum | + | <0.05 |
| B. adolescentis | + | <0.05 |
| B. bifidum | + | <0.05 |
| Ketone Bodies | + | 0.0291 |
| 3-indole propionate | + | 0.01 |
| 3-Hydroxybutyrate | + | <0.05 |
| 12,13-diHODE | + | <0.05 |
| 3-hydroxydecanoic acid | + | <0.05 |
| 4-cresol sulfate | − | <0.05 |
| Indoxyl sulfate | − | <0.05 |

### Example 2

In-vitro batch fermentation experiment was set up to compare the effect of 24 h fermentation of GOS or the active composition on the microbial metabolism in healthy adults. For this, the active composition according to Table 3 was subjected to a simulation of upper gastrointestinal tract according to the INFOGEST 2.0 method (Brodkorb et al) to mimic the human upper gastro-intestinal tract digestion before being introduced in the fermenters. This step removed small sugars such as lactose. On the other hand, GOS which is not digested by human gut was directly introduced in the fermenters. The total amount of GOS in the fermenters was the same between the two conditions (see Table 4). A negative control was also introduced to evaluate the basal microbial metabolism after 24 h without any substrate addition (NSC). Each condition was tested using fecal inoculum of 12 healthy adult donors. Microbial metabolites (including short chain fatty acids) were measured using a combination of targeted and untargeted metabolomics approaches by ultra-high performance liquid chromatography mass spectrometry (UPLC-MS).

TABLE 3
Active composition

| | Composition g/100 g |
|---|---|
| | dry matter |
| lactose | 5.7 |
| glucose | 11 |
| galactose | 1 |
| GOS | 27 |
| Protein | 35 |
| Fat | 1 |

TABLE 4
Lactose and GOS levels of compositions after INFOGEST

| | | | Composition after |
|---|---|---|---|
| | NSC-24 h | GOS-24 h | INFOGEST “MLG-H 24 h” |
| Lactose (g/L) | 0 | 0 | 0 |
| GOS (g/L) | 0 | 4.31 | 4.3 |

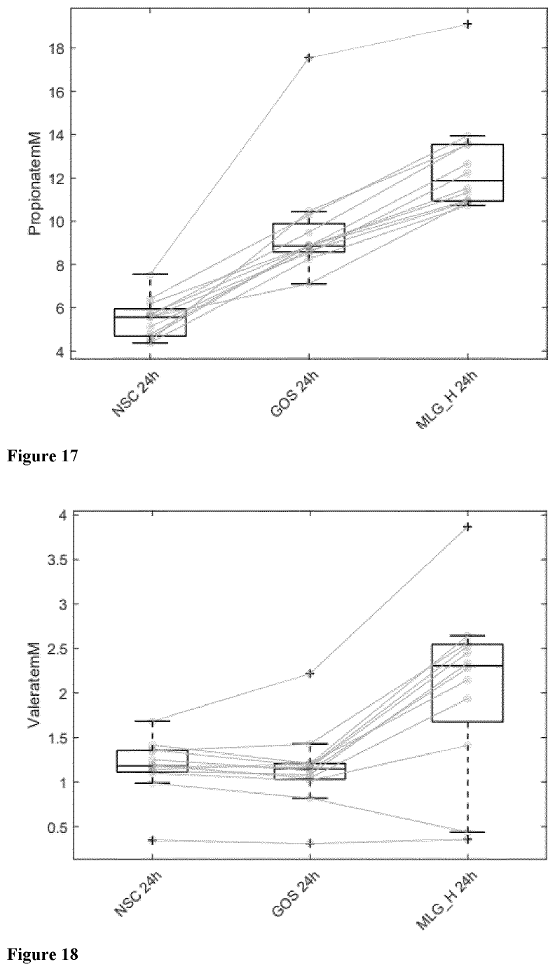

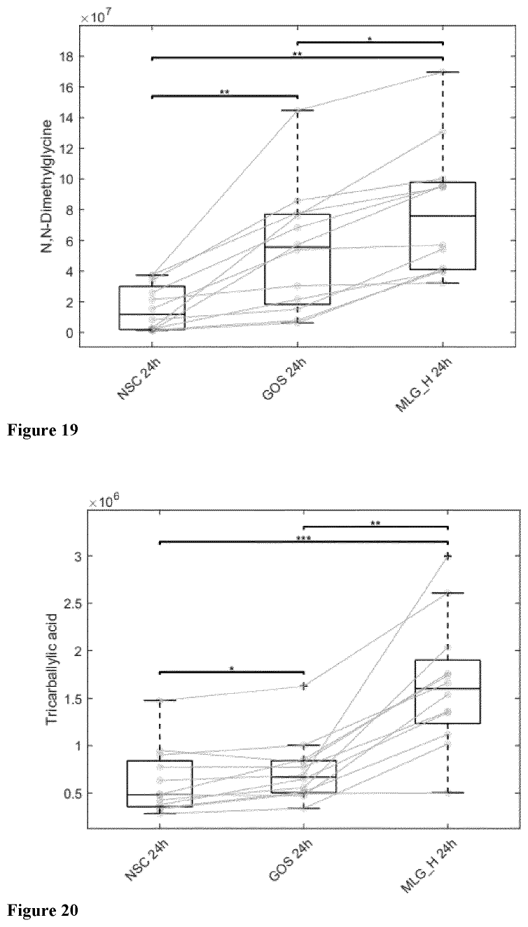

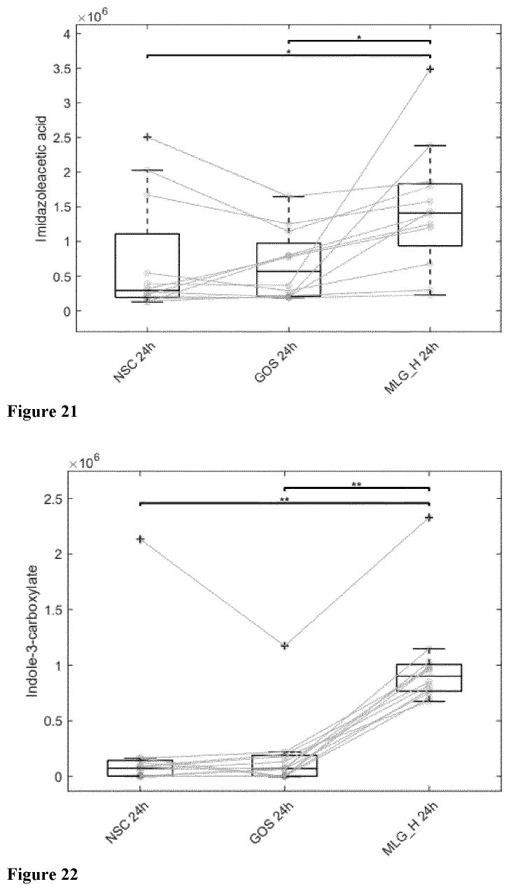

FIGS. 17-22 include box plots showing significant increase or decrease in production of beneficial metabolites after 24 h fermentation of the composition disclosed herein by comparison to GOS alone or negative control (NSC) using batch fermentation.

### EMBODIMENTS

Embodiment 1: A method for improving metabolic health, the method comprising administering to an individual in need thereof a low sugar, low lactose, milk-based composition comprising galactooligosaccharide.

Embodiment 2: The method of embodiment 1, wherein the milk-based composition comprises:
milk protein, preferably at 20-40% by dry weight of the composition; and
a mixture of lactose, glucose, galactose and galactooligosaccharide, the mixture preferably at 35-55% by dry weight of the composition;
preferably, the mixture comprising: 0.1-30% by weight of the lactose, 8-40% by weight of the glucose, 0.1-20% by weight of the galactose and 40-90% by weight of the galactooligosaccharide.

Embodiment 3: The method of embodiment 1 or embodiment 2, wherein the improving of metabolic health comprises at least one result selected from the group consisting of: (a) appetite or food intake control; (b) managing glucose levels; (c) weight management; (d) normalizing blood lipid profiles; (e) skin health; (f) reducing inflammation; (g) preventing and/or attenuating oxidative stress; (h) improved lipid metabolism; (i) improving intestinal barrier; (j) reducing intestinal permeability; (k) modulation of glucose metabolism; (l) protection from fatty liver; (m) promoting growth of Faecalibacterium prausnitzii, (n) protecting kidneys, particularly in chronic kidney disease; (o) promoting growth of Bifidobacterium; (p) increasing Bifidobacterium shunt pathway; (q) increasing NAD salvage pathway; (r) preservation of β-cell function and (s) combinations thereof.

Embodiment 4: The method of any of embodiments 1 to 3, wherein the composition is administered in an amount effective to increase at least one metabolite selected from the group consisting of: 3-indole propionate, acetate, caprylic acid, nicotinamide, beta-alanine, ketone bodies, 3-hydroxybutyrate, 12,13-diHODE, and/or 3-hydroxydecanoic acid.

Embodiment 5: The method of any of embodiments 1 to 4, wherein the composition is administered in an amount effective to increase one or more Bifidobacterium, including but not limited to B. longum, B. adolescentis, B. bifidum.

Embodiment 6: A method for one or more of preventing oxidative stress, preventing and/or treating type II diabetes, reducing inflammation, improving the intestinal barrier, reducing intestinal permeability, modulating glucose metabolism, and/or protecting against fatty liver, the method comprising administering to an individual in need thereof or at risk thereof a composition comprising:
milk protein, preferably at 20-40% by dry weight of the composition; and
a mixture of lactose, glucose, galactose and galactooligosaccharide, the mixture preferably at 35-55% by dry weight of the composition;
preferably, the mixture comprising: 0.1-30% by weight of the lactose, 8-40% by weight of the glucose, 0.1-20% by weight of the galactose and 40-90% by weight of the galactooligosaccharide.

Embodiment 7: The method of embodiment 6, wherein the composition is administered in an amount effective for increasing levels of 3-indole propionate in the individual.

Embodiment 8: The method of any of embodiments 6 and 7, wherein the composition is administered to the individual in a unit dosage form, wherein the unit dosage form comprises an amount of the composition effective for increasing levels of 3-indole propionate in an individual.

Embodiment 9: A method for preventing and/or treating kidney disease, the method comprising administering to an individual in need thereof or at risk thereof a composition comprising:
milk protein, preferably at 20-40% by dry weight of the composition; and
a mixture of lactose, glucose, galactose and galactooligosaccharide, the mixture preferably at 35-55% by dry weight of the composition;
preferably, the mixture comprising: 0.1-30% by weight of the lactose, 8-40% by weight of the glucose, 0.1-20% by weight of the galactose and 40-90% by weight of the galactooligosaccharide.

Embodiment 10: The method of embodiment 9, wherein the composition is administered in an amount effective for increasing levels of 3-indole propionate in an individual and decreased levels of at least one metabolite selected from the group consisting of p-cresol sulfate and indoxyl sulfate.

Embodiment 11: The method of any of embodiments 9 and 10, wherein the composition is administered to the individual in a unit dosage form, wherein the unit dosage form comprises an amount of the composition effective for increasing levels of 3-indole propionate and decreasing levels of at least one of metabolite selected from the group consisting of p-cresol sulfate and indoxyl sulfate in the individual.

Embodiment 12: A method for one or more of treating or preventing pellagra, acne, or skin cancer, improving skin health, and/or improving the level of blood lipids, the method comprising administering to an individual in need thereof or at risk thereof a composition comprising:
milk protein, preferably at 20-40% by dry weight of the composition; and
a mixture of lactose, glucose, galactose and galactooligosaccharide, the mixture preferably at 35-55% by dry weight of the composition;
preferably, the mixture comprising: 0.1-30% by weight of the lactose, 8-40% by weight of the glucose, 0.1-20% by weight of the galactose and 40-90% by weight of the galactooligosaccharide.

Embodiment 13: The method of embodiment 12, wherein the composition is administered in an amount effective for increasing levels of nicotinamide in the individual.

Embodiment 14: The method of any of embodiments 12 and 13, wherein the composition is administered to the individual in a unit dosage form, wherein the unit dosage form comprises an amount of the composition effective for increasing levels of nicotinamide in an individual.

Embodiment 15: A method for one or more of preventing and/or treating obesity, improving appetite control, improving food intake control, and/or managing weight, the method comprising administering to an individual in need thereof or at risk thereof a composition comprising:
milk protein, preferably at 20-40% by dry weight of the composition; and
a mixture of lactose, glucose, galactose and galactooligosaccharide, the mixture preferably at 35-55% by dry weight of the composition;
preferably, the mixture comprising: 0.1-30% by weight of the lactose, 8-40% by weight of the glucose, 0.1-20% by weight of the galactose and 40-90% by weight of the galactooligosaccharide.

Embodiment 16: The method of embodiment 15, wherein the composition is administered in an amount effective for increasing levels of acetate in the individual.

Embodiment 17: The method of any of embodiments 15 and 16, wherein the composition is administered to the individual in a unit dosage form, wherein the unit dosage form comprises an amount of the composition effective for increasing levels of acetate in an individual

Embodiment 18: A method for one or more of preventing and/or treating obesity, improving appetite control, improving food intake control, and/or managing weight, the method comprising administering to an individual in need thereof or at risk thereof a composition comprising:
milk protein, preferably at 20-40% by dry weight of the composition; and
a mixture of lactose, glucose, galactose and galactooligosaccharide, the mixture preferably at 35-55% by dry weight of the composition;
preferably, the mixture comprising: 0.1-30% by weight of the lactose, 8-40% by weight of the glucose, 0.1-20% by weight of the galactose and 40-90% by weight of the galactooligosaccharide.

Embodiment 19: The method of embodiment 18, wherein the composition is administered in an amount effective for increasing levels of Bifidobacterium in the individual.

Embodiment 20: The method of any of embodiments 18 and 19, wherein the composition is administered to the individual in a unit dosage form, wherein the unit dosage form comprises an amount of the composition effective for increasing levels of Bifidobacterium in an individual.

Embodiment 21: A method for one or more of reducing and/or preventing oxidative stress and/or treating and/or preventing age-associated diseases, the method comprising administering to an individual in need thereof or at risk thereof a composition comprising:
milk protein, preferably at 20-40% by dry weight of the composition; and
a mixture of lactose, glucose, galactose and galactooligosaccharide, the mixture preferably at 35-55% by dry weight of the composition;
preferably, the mixture comprising: 0.1-30% by weight of the lactose, 8-40% by weight of the glucose, 0.1-20% by weight of the galactose and 40-90% by weight of the galactooligosaccharide.

Embodiment 22: The method of embodiment 21, wherein the composition is administered in an amount effective for increasing levels of 3-hydroxybutyrate in the individual.

Embodiment 23: The method of any of embodiments 21 and 22, wherein the composition is administered to the individual in a unit dosage form, wherein the unit dosage form comprises an amount of the composition effective for increasing levels of 3-hydroxybutyrate in an individual.

Embodiment 24: A method for improving lipid metabolism, the method comprising administering to an individual in need thereof or at risk thereof a composition comprising:
milk protein, preferably at 20-40% by dry weight of the composition; and
a mixture of lactose, glucose, galactose and galactooligosaccharide, the mixture preferably at 35-55% by dry weight of the composition;
preferably, the mixture comprising: 0.1-30% by weight of the lactose, 8-40% by weight of the glucose, 0.1-20% by weight of the galactose and 40-90% by weight of the galactooligosaccharide.

Embodiment 25: The method of embodiment 24, wherein the composition is administered in an amount effective for increasing levels of 12,13-diHODE in the individual.

Embodiment 26: The method of any of embodiments 24 and 25, wherein the composition is administered to the individual in a unit dosage form, wherein the unit dosage form comprises an amount of the composition effective for increasing levels of 12,13-diHODE in an individual.

1: A method for improving metabolic health, the method comprising administering to an individual in need thereof a low sugar, low lactose, milk-based composition comprising galactooligosaccharide.

2: The method of claim 1, wherein the milk-based composition comprises:
milk protein; and
a mixture of lactose, glucose, galactose and galactooligosaccharide, the mixture preferably at 32-55% by dry weight of the composition.

3: The method of claim 1, wherein the improving of metabolic health comprises at least one result selected from the group consisting of: (a) appetite or food intake control or regulation; (b) managing glucose levels; (c) weight management; (d) normalizing blood lipid profiles; (e) skin health; (f) reducing inflammation; (g) preventing and/or attenuating oxidative stress; (h) improved lipid/fat metabolism; (i) improving intestinal barrier; (j) reducing intestinal permeability; (k) modulation of glucose metabolism; (l) protection from fatty liver; (m) promoting growth of Faecalibacterium prausnitzii, (n) protecting kidneys, particularly in chronic kidney disease; (o) promoting growth of Bifidobacterium; (p) increasing Bifidobacterium shunt pathway; (q) increasing NAD salvage pathway (r) preservation of β-cell function; (s) preventing diabetes, (t) improving insulin sensitivity; (u) preventing and/or treating obesity; (x) regulating blood pressure; (y) maintain insulin response; and (z) combinations thereof.

4: The method of claim 1, wherein the milk-based composition is administered in an amount effective to increase at least one metabolite selected from the group consisting of: 3-indole propionate, propionate valerate, tricarballylic acid, imidazole acetate, N,N,-dimethylglycine, indole-3-carboxylate, caprylic acid, nicotinamide, beta-alanine, ketone bodies, 3-hydroxybutyrate, 12,13-diHODE, and/or 3-hydroxydecanoic acid.

5: The method of claim 1, wherein the milk-based composition is administered in an amount effective to increase one or more Bifidobacterium.

6: A method for one or more of preventing oxidative stress, preventing and/or treating type II diabetes, reducing inflammation, improving the intestinal barrier, reducing intestinal permeability, modulating glucose metabolism, and/or protecting against fatty liver, the method comprising administering to an individual in need thereof or at risk thereof a composition comprising:
milk protein; and
a mixture of lactose, glucose, galactose and galactooligosaccharide.

7: The method of claim 6, wherein the composition is administered in an amount effective for increasing levels of 3-indole propionate in the individual.

8: The method of claim 6, wherein the composition is administered to the individual in a unit dosage form, wherein the unit dosage form comprises an amount of the composition effective for increasing levels of 3-indole propionate in the individual.

9: A method for preventing and/or treating kidney disease, the method comprising administering to an individual in need thereof or at risk thereof a composition comprising:
milk protein; and
a mixture of lactose, glucose, galactose and galactooligosaccharide.

10: The method of claim 9, wherein the composition is administered in an amount effective for increasing levels of 3-indole propionate in an individual and decreased levels of at least one metabolite selected from the group consisting of p-cresol sulfate and indoxyl sulfate.

11: The method of claim 9, wherein the composition is administered to the individual in a unit dosage form, wherein the unit dosage form comprises an amount of the composition effective for increasing levels of 3-indole propionate and decreasing levels of at least one of metabolite selected from the group consisting of p-cresol sulfate and indoxyl sulfate in the individual.

12: A method for one or more of treating or preventing pellagra, acne, or skin cancer, improving skin health, and/or improving the level of blood lipids, the method comprising administering to an individual in need thereof or at risk thereof a composition comprising:
milk protein; and
a mixture of lactose, glucose, galactose and galactooligosaccharide.

13: The method of claim 12, wherein the composition is administered in an amount effective for increasing levels of nicotinamide in the individual.

14: The method of claim 12, wherein the composition is administered to the individual in a unit dosage form, wherein the unit dosage form comprises an amount of the composition effective for increasing levels of nicotinamide in an individual.

15: A method for one or more of preventing and/or treating obesity, improving appetite control, improving food intake control, and/or managing weight, the method comprising administering to an individual in need thereof or at risk thereof a composition comprising:
milk protein; and
a mixture of lactose, glucose, galactose and galactooligosaccharide.

16: The method of claim 15, wherein the composition is administered in an amount effective for increasing levels of acetate in the individual.

17: The method of claim 15, wherein the composition is administered to the individual in a unit dosage form, wherein the unit dosage form comprises an amount of the composition effective for increasing levels of acetate in the individual.

18: A method for one or more of preventing and/or treating obesity, improving appetite control, improving food intake control, and/or managing weight, the method comprising administering to an individual in need thereof or at risk thereof a composition comprising:
milk protein; and
a mixture of lactose, glucose, galactose and galactooligosaccharide.

19: The method of claim 18, wherein the composition is administered in an amount effective for increasing levels of Bifidobacterium in the individual.

20: The method of claim 18, wherein the composition is administered to the individual in a unit dosage form, wherein the unit dosage form comprises an amount of the composition effective for increasing levels of Bifidobacterium in the individual.

21: A method for one or more of reducing and/or preventing oxidative stress and/or treating and/or preventing age-associated diseases, the method comprising administering to an individual in need thereof or at risk thereof a composition comprising:
milk protein; and
a mixture of lactose, glucose, galactose and galactooligosaccharide.

22: The method of claim 21, wherein the composition is administered in an amount effective for increasing levels of 3-hydroxybutyrate in the individual.

23: The method of claim 21, wherein the composition is administered to the individual in a unit dosage form, wherein the unit dosage form comprises an amount of the composition effective for increasing levels of 3-hydroxybutyrate in the individual.

24: A method for improving lipid metabolism, the method comprising administering to an individual in need thereof or at risk thereof a composition comprising:
milk protein; and
a mixture of lactose, glucose, galactose and galactooligosaccharide.

25: The method of claim 24, wherein the composition is administered in an amount effective for increasing levels of 12,13-diHODE in the individual.

26: The method of claim 24, wherein the composition is administered to the individual in a unit dosage form, wherein the unit dosage form comprises an amount of the composition effective for increasing levels of 12,13-diHODE in the individual.
